(19) United States
(12) Patent Application Publication
(10) Pub. No.: US 2026/0000873 A1
(43) Pub. Date: Jan. 1, 2026

(54) IV CATHETER DEVICE

(71) Applicant: NEW YORK SOCIETY FOR THE RELIEF OF THE RUPTURED AND CRIPPLED, MAINTAINING THE HOSPITAL FOR SPECIAL, New York, NY (US)

(72) Inventor: Michael Singleton, New York, NY (US)

(73) Assignee: NEW YORK SOCIETY FOR THE RELIEF OF THE RUPTURED AND CRIPPLED, MAINTAINING THE HOSPITAL FOR SPECIAL, New York, NY (US)

(21) Appl. No.: 18/992,826

(22) PCT Filed: Jul. 12, 2023
(86) PCT No.: PCT/US2023/027472

§ 371 Date: Jan. 9, 2025

Related U.S. Application Data
(60) Provisional application No. 63/388,747, filed on Jul. 13, 2022.

(51) Int. Cl. A61M 25/06; A61M 25/01

(52) U.S. Cl. CPC A61M 25/0606; A61M 25/0113; A61M 25/0693

(57) ABSTRACT

An IV catheter device includes a needle component having an elongated needle with a sharp distal tip and a hollow needle hub that defines a flash chamber. The device includes a catheter component having a hollow body through which the needle passes and an actuatable obturator that is disposed within the hollow needle hub and moves between a pre-deployment position and a deployed position. In both the pre-deployment position and the deployed position, the actuatable obturator is disposed within an inner lumen of the elongated needle. In the deployed position, a blunt distal end of the obturator blunts the elongated needle. The actuatable obturator is biased by a biasing element that releases stored energy as the actuatable obturator moves from the pre-deployment position to the deployed position. The biasing element is disposed within the hollow needle hub within the flash chamber. The actuatable obturator is hollow and has an inner lumen formed therein that is open at proximal and distal ends of the obturator, with the inner lumen at the proximal end of the obturator being open to an area of the flash chamber that contains the biasing element.

### CROSS-REFERENCE TO RELATED APPLICATION

The present application claims the benefit of and priority to U.S. patent application Ser. No. 63/388,747, filed Jul. 13, 2022, which is hereby expressly incorporated by reference in its entirety.

### TECHNICAL FIELD

The present invention is directed to intravenous equipment and more particularly, to IV catheter (delivery) systems that each includes an actuatable obturator that is configured to facilitate placement of the IV catheter system within the vein by blunting a sharp needle tip of the needle that is used to guide the delivery of the IV catheter system.

### BACKGROUND

As is well understood, IV stands for intravenous and concerns delivery fluids or medicines through a needle or tube (catheter) into a vein. The needle is usually placed in a vein near the elbow, the wrist or in the back of the hand. IV fluids or medicine are delivered in a wide array of different settings. One specific application in which medicine is delivered by an IV is the field of anesthesia which uses drugs or other methods to create a loss of awareness and block feelings of pain. It increases patient comfort and safety during medical procedures. Under general anesthesia, the patient is unaware and does not sense pain. Because the patient cannot breathe without help, a breathing machine is required. A breathing tube or other airway device delivers general anesthesia and maximizes patient safety. General anesthesia uses a variety of drugs and methods. The most common method is through breathing gas after an intravenous (IV) injection. The patient breathes in gases that are absorbed by the lungs and delivered through the bloodstream to the brain and spinal cord.

An IV catheter is the primary means for delivering the IV fluid/medicine to the patient. Intravenous (IV) cannulation is a technique in which a cannula (IV catheter) is placed inside a vein to provide venous access. Venous access allows sampling of blood, as well as administration of fluids, medications, parenteral nutrition, chemotherapy, and blood products.

Veins have a three-layered wall composed of an internal endothelium surrounded by a thin layer of muscle fibers that is surrounded by a layer of connective tissue. Venous valves encourage unidirectional flow of blood and prevent pooling of blood in the dependent portions of the extremities; they also can impede the passage of a catheter through and into a vein.

An IV catheter is introduced into the vein by a needle and then is fixed by being taped to a patient's skin. Most modern day IV catheters are equipped with a safety mechanism to shield the user from the needle as the needle is exposed and then later removed and retracted.

IV cannulation is typically performed by completing the following steps:
(1) a good vein is found to insert the IV catheter (e.g., a large vein located on the underside of the forearm);
(2) apply a tourniquet just a few inches above the site to get your veins to swell up for easy IV catheter insertion;
(3) disinfect the area with an alcohol pad. Wipe gently but thoroughly, ensuring an even coat of alcohol. This minimizes the chance of infection;
(4) use the right sized catheter. Ensure the needle doesn't touch anything other than the patient's skin. This can compromise their sterility and increase the risk of infection.
(5) stabilize the patient's limb with gentle pressure, taking care not to touch the IV site directly;
(6) remove the cap on the catheter and tightly pull the skin around the IV site. Insert the needle with minimum angle (as parallel to the skin as possible);
(7) the flashback of blood in the catheter's applicator will indicate that the vein has been hit, advance the needle one more centimeter (cm) into the vein;
(8) pull the needle back about 1 centimeter out of the vein. When the cannula is seated in the vein, remove the tourniquet and put a sterile bandage over the lower half of the catheter hub;
(9) dispose the needle for the safety of everyone involved and remove the tourniquet to prevent discomfort; and
(10) finally apply a piece of tape to the area around the catheter while attaching the IV tubing. Ensure there are no twists in the loop.

Unfortunately, there has been little improvement on the placement of an IV catheter over the years and most advancements have been in improvement in IV safety (prevention of needle sticks). There have been minimal design features to help novices achieve high rates of first attempt success when inserting and starting an IV. As mentioned, once the sharp tip of needle (beveled tip) enters vein, a flash of blood (a flashback) is seen in a chamber visible to the practitioner. However, when the tip of the needle enters the vein, the tip of catheter is still set back a short distance from the needle point. If a practitioner attempts to advance the catheter before the entire tip of the catheter is in the vein as well, it will not advance and will possibly disrupt and jeopardize the integrity of the vein. To prevent this from happening, once the flash is seen, the practitioner, must advance the needle further forward (a distance which depends on how big the needle is and how far the catheter tip is from the needle point) until the catheter is in the vein. This additional advancement runs the risk of “back-walling” the vein, or the needle point puncturing the other side of the vein and thus disrupting the integrity of the vein.

There is therefore a need for an IV catheter device that addresses and overcomes the above noted deficiencies and one that allows for proper placement of the catheter without the risk of “back-walling” the vein.

### SUMMARY

An IV catheter device according to one embodiment includes a needle component having an elongated needle with a sharp distal tip and a catheter component having a hollow body through which the needle passes. The IV catheter device further includes an actuatable obturator that is disposed within the needle component and moves between a pre-deployment position and a deployed position. In both the pre-deployment position and the deployed position, the actuatable obturator is disposed within an inner lumen of the elongated needle. In the deployed position, a blunt distal end of the obturator is located distal to the sharp distal tip so as to blunt the elongated needle. The actuatable obturator is biased by a biasing element that stores energy in the pre-deployment position and releases energy as the actuatable obturator moves from the pre-deployment position to the deployed position. The biasing element is disposed within the hollow needle hub within the flash chamber. The actuatable obturator is hollow and has an inner lumen formed therein that is open at proximal and distal ends of the obturator, with the inner lumen at the proximal end of the obturator being open to an area of the flash chamber that contains the biasing element.

The present disclosure also sets forth a method for selectively blunting a sharp distal end of a needle that is part of an IV catheter device comprising the step of: actuating an obturator that is part of an IV catheter device to cause the obturator to travel within an inner lumen of the needle until a blunt distal end of the obturator is located distal to the sharp distal end of the needle so as to blunt the needle.

### BRIEF DESCRIPTION OF THE DRAWING FIGURES

FIG. 1 is a side elevation view of an IV catheter device with an obturator according to one exemplary embodiment;

FIG. 2 is a side perspective view thereof;

FIG. 3 is an exploded perspective view thereof;

FIG. 4 is a side cross-sectional view showing a pre-deployed position of the IV catheter device;

FIG. 5 is a side cross-sectional view showing a deployed position of the IV catheter device;

FIG. 6 is a close-up of a distal end of the IV catheter device;

FIG. 7 is a close-up cross-sectional view of a portion of the IV catheter device;

FIG. 8 is a perspective view of an obturator actuator; and

FIG. 9 is a side elevation view thereof.

### DETAILED DESCRIPTION OF CERTAIN EMBODIMENTS

In accordance with the present disclosure, improved IV catheter devices described and illustrated in the appended figures. It will be understood that components/elements that are used and present in multiple embodiments are numbered alike.

### IV Catheter Construction 100

An IV catheter (device) in accordance with a first embodiment is illustrated by reference character 100.

The IV catheter 100 includes a number of components that mate together and are coupled to one another to form an assembled product as shown in the figures. More particularly, the IV catheter 100 includes a catheter component 110, a needle component 120, a main housing (handle) 130 and an obturator 200. An actuator 300 is also preferably provided to permit a user to selectively activate the obturator 200.

### Catheter component 110

The catheter component 110 has a hollow main catheter body that has a first (distal) end and an opposing second (proximal) end. The main catheter body includes two parts or regions, namely, an elongated hollow cannula 112 and a hub portion 114. The elongated hollow cannula 112 extends from and distal to the hub portion 114. The hub portion 114 itself is a hollow part that and thus the hollow interiors of the cannula 112 and the hub portion 114 are in fluid communication with one another. As shown, the hub portion 114 is traditionally larger in size than the hollow cannula 112.

The hub portion 114 can have a stepped construction both along the exterior and the interior. Internally between the different regions of the hub portion 114 that have different inner diameters, an inner shoulder 115 can be defined and can define one end of travel within the inner lumen.

The distal end of the hollow cannula 112 can be a blunt end.

The main catheter body, and in particular, the hub portion 114 can have one or more wings or side protrusions (flanges or tabs) that extend radially outward from the main catheter body (the hub portion 114). The side protrusions serve several different purposes in that they provide surfaces for grasping and manual handling of the IV catheter component for advancing it into the vein. In addition, the side protrusions provide surfaces for securing the IV catheter component 110 to the patient's body as by using an adhesive tape or the like. For example, each side protrusion can have a piece of tape that is attached thereto and extends beyond edges for the side protrusion for attaching to the skin. Alternatively, the outer surface can be substantially smooth.

The elongated hollow cannula 112 is the portion of the catheter component 110 that is driven into a vein, while the larger main catheter body (the hub portion 114) remains outside the patient's body.

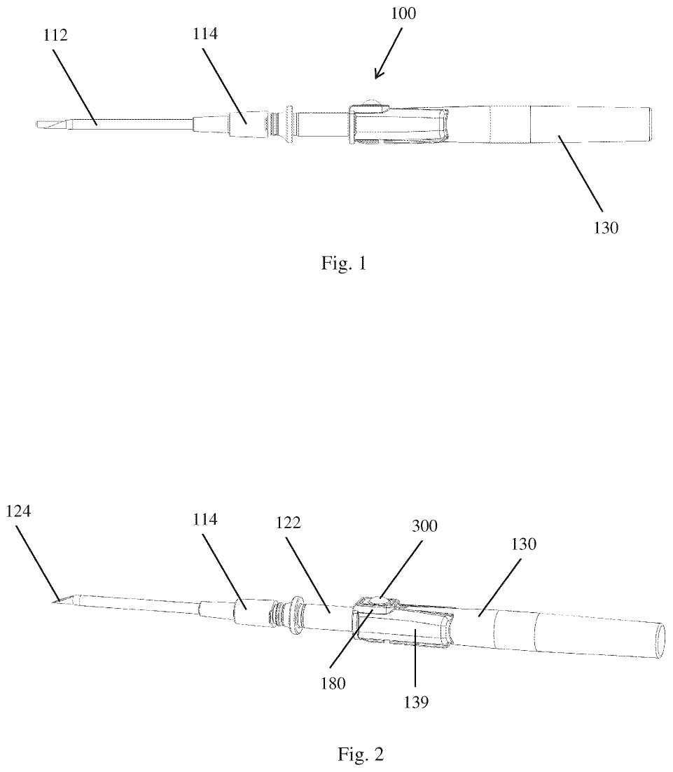

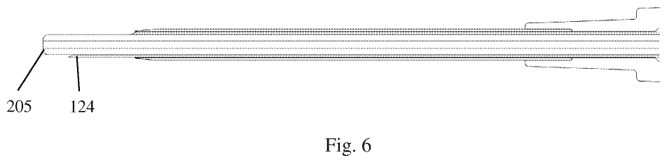

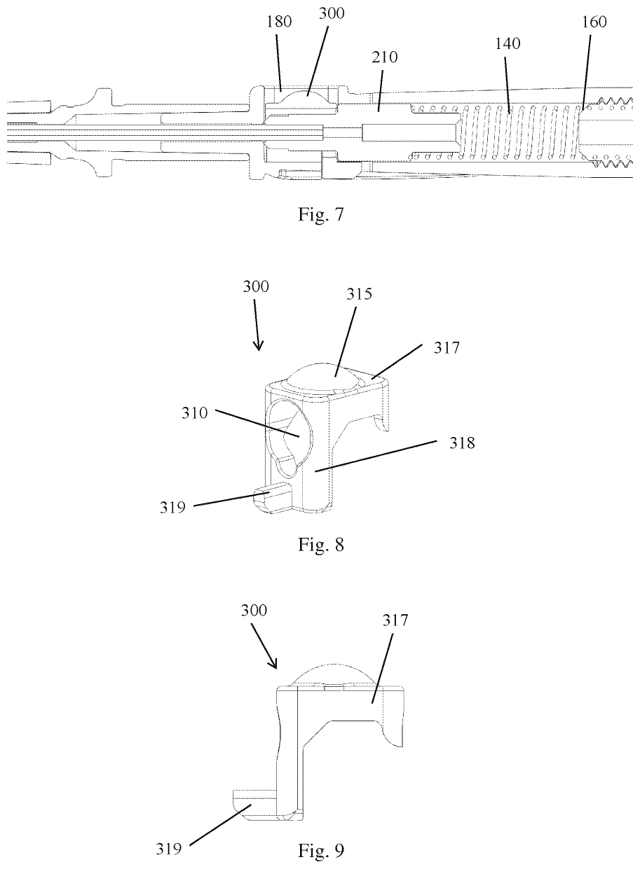

The catheter component 110 can include a valve to allow for injection of fluid (e.g., drugs) with a syringe. Any number of suitable valves can be used. Alternatively, the catheter component 110 can have a simple construction with no valve as shown in FIGS. 1-9.

The proximal end of the catheter component 110 is the end which allows for subsequent connection to an intravenous infusion line (tubing) and capping in between uses. As is known, the infusion line is placed in fluid communication with the main catheter body 110 for delivering fluid from a source, such as an IV bag or the like, to the vein through the cannula 112.

### Needle Component 120

The sharp needle component 120 serves as a guidewire for inserting the cannula (i.e., the distal tip of the hollow cannula 112) into the vein. As shown, the needle component 120 is a separate part from the IV catheter component 110.

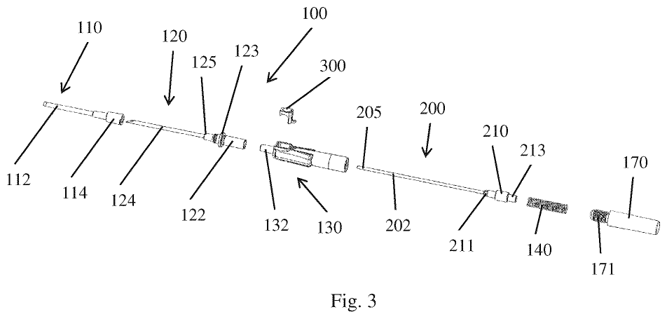

In FIG. 3, the needle component 120 includes a needle hub portion 122 for grasping the needle component 120 and includes, of course, an elongated hollow needle 124. The hub portion 122 is much wider than the needle and a shoulder 125 is formed at the end of the hub portion 122 where the needle is coupled. The elongated hollow needle 124 has a sharp distal end.

As shown, the hollow hub portion 122 has a non-uniform inner diameter with the distal end of the hollow hub portion 122 being less than the inner diameter at the proximal end of the hollow hub portion 122.

Along an exterior of the hollow hub portion 122, a flange 123 can be provided. The flange 123 is intended to allow the user to move the needle component 120 relative to the catheter component 110 (e.g., attach or detach the needle component 120 from the catheter component 110).

In one embodiment, the needle component 120 can be received within the catheter component 110 and more particularly, the hub portion 122 is received within the hollow portion 114 of the catheter component 110. For example, a friction fit can be formed between these two parts.

More specifically, the hollow needle 124 is sized to be received within the elongated hollow cannula 112 of the catheter component 110 and can slide within the elongated hollow cannula 112 to allow the sharp distal end of the hollow needle 124 to extend beyond the blunt distal end of the hollow cannula 112 of the catheter component 110 when the needle component 120 is securely attached to the catheter component 110 as be reaching an end of travel of the needle component 120 relative to the catheter component 110.

The hollow needle 124 is typically formed of a metal, while the hollow hub portion 122 is formed of a plastic material.

### Actuatable Obturator 200

As described herein, in accordance with the present disclosure, the actuatable obturator 200 functions to selectively transform the sharp distal tip of the hollow needle 124 into a blunt end after the hollow needle is inserted into the vein.

In the embodiment shown in FIGS. 1-9, the actuatable obturator 200 is hollow and includes dimensions that allow the obturator 200 to be advanced through the lumen of the elongated hollow needle 124 and exit the distal tip of the hollow needle 124. The obturator 200 thus comprises an elongated hollow structure 202, such as a hollow tube or the like, that is advanced through the lumen of the hollow needle 124. The diameter of the elongated structure 202 is thus the same as or slightly less than the diameter of the lumen of the needle so that the obturator 200 is in close proximity or in contact with the inner wall of the needle that defines the inner lumen. The elongated structure 202 terminates in a distal blunt end

The obturator 200 also includes a hub portion 210 to which the elongated hollow structure 202 is coupled or is integral thereto. The elongated hollow structure 202 thus extends distally from the hub portion 210. The hub portion 210 is thus an enlarged part compared to the elongated structure 202. The hub portion 210 is a hollow structure and thus the obturator 200 is open at both its ends with one end forming an entrance into the hollow structure 202. As shown, the lumen through the hub portion 210 can have a varying diameter with the lumen having a greater diameter at a proximal end of the hub portion 210. A portion of the elongated hollow structure 202 is anchored in the hub portion 210 or is otherwise attached thereto.

The hub portion 210 has a distal end region 211 and a proximal end region 213.

The hub portion 210 has an outer surface and the hub portion 210 can have a first region or portion that has increased dimensions. For example, this region can be a center section of the hub portion 210 located between regions 211, 213. As described below, an outer (exterior) surface of the first region can be intended to seat and seal against the inner surface of the main housing 130.

The proximal end region 213 of the hub portion 210 can have a diameter less than the center region and thus an outer shoulder 217 is formed between the wider center region and the proximal end region 213.

The elongated structure 202 and the hub portion 210 can be formed of the same material or different materials.

### Main Housing 130

In the embodiment shown in FIGS. 1-9, the main housing 130 is a separate part relative to the needle component 120; however, in other embodiments, the main housing 130 and the needle component 120 (e.g., at least the hub portion thereof) can be combined into a single part. When combined, the single structure can be referred to as a needle component.

The main housing 130 serves several purposes in that it contains a portion of the actuatable obturator 200, contains a biasing element (e.g., spring), and contains the actuator 300 that is configured to controllably release the actuatable obturator 200.

The main housing 130 has a distal end that engages the needle component 120 and has an opposite proximal end. Like the other components, the main housing 130 is a hollow part that is open at both the distal end and the proximal end.

The distal end of the main housing 130 is configured to be received within and engage the proximal end of the needle component 120 when the two are separate parts. For example, the distal end of the main housing 130 can include a finger or boss 132 that has a smaller outer diameter than the other portions of the main housing 130. The hollow boss 132 can be received and held within the hollow proximal end (hub portion 122) of the needle component 120 as by a friction fit or by other means, such as a snap-fit. In other words, the outer diameter of the hollow boss 132 is about equal to the inner diameter of the hollow hub portion 122 of the needle component 120.

An outer shoulder 133 is formed at the interface between the finger 132 and the adjacent portion of the main housing. This outer shoulder 133 acts as a stop in that when the main housing 130 is fully inserted into the needle component 120, the proximal end of the needle component 120 seats against the outer shoulder 133.

The main housing 130 includes a biasing member 140 that is disposed within the hollow main housing and applies a biasing force against the hub portion 210 of the obturator 200 to cause controlled forward advancement of the obturator 210. The biasing member 140 can be a coiled spring as shown. The outer diameter of the coiled spring can be the same or very close to the inner diameter of the main housing 130 and this results in the coiled spring seating against or close to the inner wall of the main housing 130.

The coiled spring 140 is sized so that it can receive the proximal end region 213 of the hub portion 210. The reception of the distal end of the coiled spring around the proximal end region 213 of the hub portion couples the coiled spring to the obturator 200. A first (distal) end of the coiled spring seats against the outer shoulder 217, while an opposing second (proximal) end of the coiled spring seats against a stop member 160 that is part of or associated with the main housing 130 (or end cap 170). A portion of the coiled spring thus surrounds the portion 213.

This stop member 160 can be a local fixed area of the main housing 130 that has a reduced diameter (or the distal end of the end cap 170) so that the proximal end of the coiled spring seats against the stop member 160. Thus, one end of the coiled spring seats against a movable objection (i.e., the obturator 200), while the other end of the coiled spring seats against a fixed, non-movable part. Thus, when the obturator 200 moves in a proximal direction toward the stop member 160, the coiled spring compresses and stores energy. When the coiled spring stores energy and the obturator 200 is released from its locked position, the obturator 200 is propelled forward to its deployed position in which the blunt distal end of the obturator 200 extends beyond the sharp distal end 205 of the needle 124.

For purpose of illustration only, the main housing 130 is shown as containing two sections including a hollow end cap 170; however, it will be appreciated that the end cap 170 can be eliminated and the main housing 130 can be a single structure that contains and surrounds the working components of the device. In the event that an end cap 170 is used, the end cap 170 mates with the remaining part of the main housing 130 and closes off the hollow interior of the main housing. The illustrated prototype shows a hollow end cap 170 with a proximally located threaded boss 171 (with outer threads) that can mate with inner threads associated with the main housing 130 to combine the two parts into one assembly. However, as mentioned, in other embodiments, the end cap 170 is eliminated and the main housing 130 is a single part that can be formed to encapsulate the working components. Different known manufacturing techniques can be used to form the main housing 130 around the working components.

As shown, the flash chamber can be at least partially located in the hollow end cap 170.

The stop 160 can be one end of the threaded boss 171 when the end cap 170 is present. In cases in which the end cap 170 is not present, the stop 160 can be one or more tabs, flanges or ridges that are formed internally within the main housing 130 and provide a surface against which one end of the coiled spring seats since the stop 160 is located radially inward to the coiled spring to prevent the coiled spring from moving in a proximal direction.

The main housing 130 can be thought of as including at least three regions, a distal region, a center region and a proximal region. The distal region can contain the actuator 300, the center region contains the hub portion 210 and the proximal region contains the biasing member 140 and also at least partially defines the blood flash chamber.

A pair of side flanges or fingers 139 can be formed as part of the main housing 130 and in particular, the side flanges 139 are formed and extend along the cylindrical shaped main housing 130. These side flanges 139 provide grip surfaces to facilitate holding the device.

The main housing 130 also include a side through hole (window). As described below, the actuator 300 passes through and is accessible within the side through hole that is bordered by a raised edge or flange 180. The border 180 surrounds the actuator 300 to prevent accidental contact with the actuator 300.

One of the functions of the main housing 130 is that it defines the flash chamber. As described below in more detail, a blood flash enters the hollow obturator 200 and flows through the hollow hub portion 210 and exits into the hollow main housing 130 (and/or into hollow end cap 170). In fact, the flash blood exits into the region of the main housing 130 that contains the spring (biasing member). The open inner lumen of the hub portion 210 of the obturator 200 can be axially aligned with a center of the coiled spring. The flash blood is thus present within the section of the main housing 130 that is proximal to the hub portion 210 and thus, does not interfere with the forward advancement (firing) of the obturator 200. It will be understood that a portion of the flash chamber can be located proximal to the stop 160 and thus, proximal to the coiled spring 140.

As mentioned, in another embodiment, the main housing 130 and the needle component 120 can be formed as a single part in that the elongated hollow needle 124 can be attached to a distal end of the main housing 130.

The main housing 130 can be transparent and can be colorless.

### Actuator 300

The actuator 300 is designed to cause the controlled release (firing) of the obturator 200 and permits the obturator 200 to move between a pre-deployed position and a deployed position. The release (actuation) of the obturator 200 allows the biasing member (coiled spring) to release its energy and propel (advance) the obturator 200 from its pre-deployed position (proximal position) to its deployed position (distal position). There are many different types of actuators that can be used in the present device. For example, different types of buttons, sliders, etc., can be used that engage a portion of the obturator 200 for the purpose of holding the obturator 200 in a locked position which happens to be the pre-deployed position of the obturator 200. Once this interference between the actuator 300 and the obturator 200 is removed, the stored energy of the compressed coiled spring is released pushing the obturator forward to the deployed position.

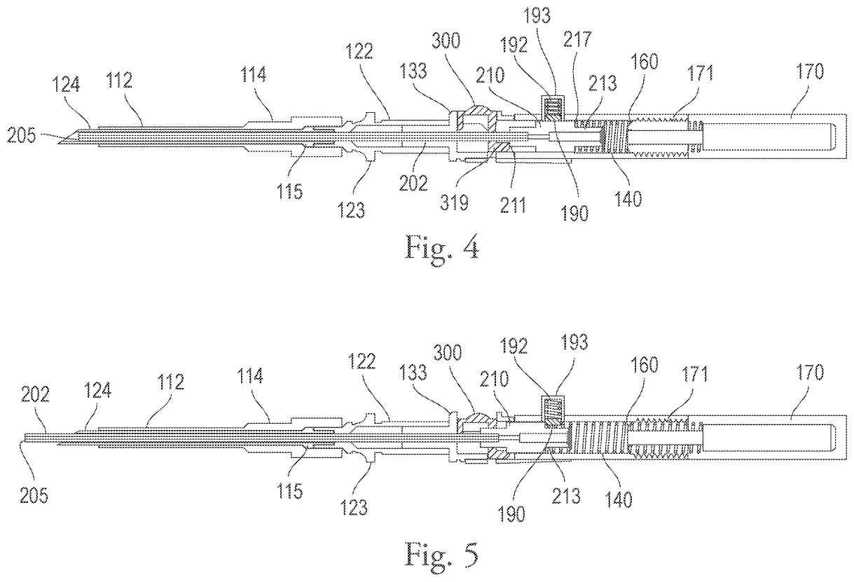

FIG. 4 shows the pre-deployed position of the obturator 200, while FIG. 5 shows the deployed position of the obturator 200.

FIGS. 8 and 9 illustrate one exemplary actuator 300. The illustrated actuator 300 has a first (vertical) wall 318, a first (upper) leg 317, and a second (lower leg) 319. As shown, the first leg 317 extends in a first direction from the first wall 318 and the second leg 319 extends in a second direction from the first wall 318 (when installed, the first leg 317 faces the needle component and the second leg 319 faces the hub portion 210 of the obturator and the coiled spring). Each of the first leg 317 and the second leg 319 is formed perpendicular to the first wall 318. Along the first leg 317, a raised portion 315 is present and this raised portion 315 defines a contact surface against which a thumb or finger can be placed to operate the actuator 300. In the illustrated embodiment, the actuator 300 operates and moves in and up and down manner. The second leg 319 is designed to engage and block the hub portion 210 of the obturator 200 and prevent forward movement thereof in the pre-deployed position. The second leg 319 thus represents a movable stop that holds the obturator in the pre-deployed position. For example, the second leg 319 can seat against the outer shoulder between the distal and center regions of the hub portion 210.

The first wall 318 includes an opening 310 that is complementary to the elongated structure 202 of the obturator 200. In both the pre-deployed and deployed positions, the elongated structure 202 passes through the opening 310.

To operate the actuator 300, the user presses down on the raised portion 315 to cause downward (linear) movement of the actuator 300, whereby the second leg 319 becomes displaced from the hub portion 210 and the obturator 200 is free to move in the distal direction under action of the biasing member (coiled spring 140). This action allows the obturator 200 to move from the pre-deployed position (FIG. 4) to the deployed position (FIG. 5). It will be seen in FIG. 5 that the actuator 300 is in a lowered position. In the deployed position, the center region of the hub obturator 210 contacts the first wall 318 of the actuator 300, while the distal end region 211 passes through the opening 310.

Once the device 100 is in the desired position, the obturator is thus fired by operating the actuator 300 (e.g., by pressing down the actuator) to remove interference between the actuator 300 and the spring biased obturator 200) to cause the obturator to move from the pre-deployed position to the deployed position, whereby the sharp distal needle tip is blunted.

Again, it will be appreciated that different types of actuators can be used that operate in different manners.

### Locking Mechanism

It will be appreciated that after deployment of the actuatable obturator 200, a mechanism can be provided to ensure that the actuatable obturator remains in the deployed position and does not back into the inner lumen of the needle after deployment (e.g., as by application of force in the proximal direction). If the actuatable obturator 200 backs into the inner lumen of the needle, the sharp tip would be exposed again.

To ensure that the actuatable obturator 200 remains in the deployed position several techniques can be used.

One technique is the use of a lock mechanism or the like which itself is operable after the actuatable obturator 200 is deployed. One type of lock mechanism can be manually operated after deployment of the actuatable obturator 200 as by the user pressing a button or the like that operates the locking mechanism. For example, a button or the like that is located along the side wall of the main housing can be operated by the user and be caused to engage the hub portion of the actuatable obturator 200. For example, a locking tab or pin can be driven into position along the proximal face of the hub portion of the actuatable obturator 200, thereby preventing unintended retraction of the actuatable obturator 200 into the inner lumen of the needle after deployment. Alternatively, the locking tab or pin can be driven into contact with a forward part of the coil spring, thereby preventing the coil spring from compressing. This arrangement thus restricts rearward movement of the obturator since the locking tab or pin acts as a stop or obstruction.

This type of lock mechanism can be configured so that the user can reverse and release the lock mechanism, thereby freeing the actuatable obturator. This would allow the manual retracting of the actuatable obturator 200 to purposefully recreate the sharp tip, but the retraction should not be from pressure applied at the forward tip.

In addition, the lock mechanism can be automated and tripped by the deployment of the actuatable obturator 200. For example, the enlarged hub portion of the actuatable obturator 200 can contact and trip a locking mechanism that then engages the hub portion, such as the proximal end of the hub portion, to create a mechanical interference that prevents retraction of the actuatable obturator within the inner lumen of the needle (i.in the proximal direction).

Suitable lock mechanisms are described in PCT/US2021/045187 ('187 application), which is hereby expressly incorporated herein by reference in its entirety. For example, FIGS. 20A-C in the '187 application show different suitable lock mechanisms.

The lock mechanism can be automated and thus can be not accessible to the user. For example, a spring biased locking pin can be provided as part of one of the main housing and obturator hub, while the other structure includes a notch or slot that receives the spring biased locking pin only when the obturator has reached its end of travel in the deployed position. Prior to reaching this deployed position, the spring biased pin is constrained and the spring compressed; however, when the obturator reaches the deployed position, the spring biased locking pin aligned with the locking notch or slot and thus, the spring leases its stored energy and the locking pin advances into the locking notch or slot to lock the obturator relative to the main housing.

For example, FIGS. 4 and 5 illustrate one exemplary automated locking mechanism that will engage without action by the user. In this embodiment, the main housing 130 includes a lock member 190 that is biased by a spring 192. Both the lock member 190 and the spring 192 can be contained within the main housing. As shown, the lock member 190 and the spring 192 are contained in a dedicated region of the main housing that can be defined by a hollow extension 193 of the main housing that extends away from the top surface of the main housing.

In FIG. 4, the lock member 190 is in a retracted state and the spring 192 is compressed and is storing energy. The lock member 190 is retracted because the hub portion 210 in the pre-deployment position urges the lock member 190 to its retracted state. The presence of the hub portion 210 in front of the lock member 190 prevents the lock member 190 from being advanced under action of the spring 192. As the obturator 200 is advanced in a distal direction from the pre-deployment position to the deployed position, the hub portion 210 moves in the distal direction, while the lock member 190 remains in a fixed location. The lock member 190 is located such that in the fully deployed position, the widest part of the hub portion 210 clears and moves distal to the lock member 190 and this allows the lock member 190 to advance downward. In particular, the lock member 190 advances downward and seats against at least a portion of the shoulder 217. The lock member 190 is located behind the shoulder 217 and thus, prevents the retraction of the obturator (movement of the obturator in the proximal direction).

### Echogenic Obturator

The obturator 200 can contain design features which allow for increased echogenicity and improved visualization when placed under ultrasound guidance. The features could contain either materials which are inherently more echogenic than the surrounding materials or entail etchings in the obturator which increase the number of ultrasound beams returning to the probe. This increased echogenicity would be used after deployment of the obturator 200 upon obtaining a flash of blood. The improved visualization of the obturator 200 would provide additional information about how far and at what orientation to advance the system into the vessel. The features may be placed at predetermined intervals to aid in determining how far into the vessel the user has advanced.

Suitable echogenic obturator construction that can be implemented with the present device are described in PCT/US2021/045187.

One or more regions of echodense material can provide increased echogenicity. The echodense regions can be provided in the form of one or more echodense bands. In particular, at or proximate the distal end (distal tip) of the obturator 200, there can be at least a first band and a second band that is longitudinally spaced from the first band. Each of the first band and the second band can comprise an etched region of the obturator or can be formed of an echodense material. The first band and the second band can be in the form of continuous etched rings that extend circumferentially about the outer surface of the obturator body. In one embodiment, the first band and the second band can be etchings in the obturator body that are at predetermined interval distances. These etchings provide increased echogenicity of the obturator 200 after deployment.

An alternative design in which instead of one or more spaced etchings, the obturator includes a solid echodense tip.

One skilled in the art would understand that there are many different types of materials that are echodense to provide visualization under traditional imaging techniques (e.g., ultrasound). Etchings provide different scattering patterns to allow for improved visualization.

As mentioned herein, the provision of one or more regions of echodense material provide for increased visualization of the obturator especially post deployment. When more than one region of echodense material is provided at spaced intervals, the degree of deployment can be determined. For example, the user can determine the degree of obturator deployment by visualization of the locations of the one or more echodense regions relative to other landmarks. For example, the width of the bands is known and therefore, the degree of which the band is visible beyond the needle tip allows the position of the obturator 200 to be easily determined.

While the applications of the present catheter devices are discussed in terms of being used in intravenous access, it will be appreciated that the present devices can be used in other applications. For example, the present devices can be used in other kinds of vascular access (intra-arterial) or other procedures where it might be useful to move from a sharp to a blunt tip, such as peripheral nerve blocks or neuraxial procedures.

In all of the embodiments described herein, the obturator can be formed of a transparent material to allow the user to see the blood flash. In addition, the hollow hub portion of the needle component is also typically transparent.

It is to be understood that like numerals in the drawings represent like elements through the several figures, and that not all components and/or steps described and illustrated with reference to the figures are required for all embodiments or arrangements.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presences of stated features, integers, steps, operations, elements, and/or components, but do not preclude the presence or addition of one or more other features, integers, steps, operations, elements, components, and/or groups thereof.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The subject matter described above is provided by way of illustration only and should not be construed as limiting. Various modifications and changes can be made to the subject matter described herein without following the example embodiments and applications illustrated and described, and without departing from the true spirit and scope of the present invention, which is set forth in the following claims.

What is claimed is:

1. An IV catheter device comprising:
a needle component having an elongated needle with a sharp distal tip and a hollow needle hub from which the elongated needle extends;
a catheter component having a hollow body through which the needle passes;
a main housing that is coupled at one end to the hollow needle hub or is integrally formed therewith, the main housing defining a flash chamber; and
an actuatable obturator that is disposed within the main housing and moves between a pre-deployment position and a deployed position, wherein in both the pre-deployment position and the deployed position, the actuatable obturator is disposed within an inner lumen of the elongated needle that is distal to the hollow needle hub, wherein in the deployed position, a blunt distal end of the obturator is located distal to the sharp distal tip so as to blunt the elongated needle;
wherein the actuatable obturator is biased by a biasing element that stores energy in the pre-deployment position and releases energy as the actuatable obturator moves from the pre-deployment position to the deployed position; the biasing element being disposed within the main housing within the flash chamber;
wherein the actuatable obturator is hollow and has an inner lumen formed therein that is open at proximal and distal ends of the obturator, the inner lumen at the proximal end of the obturator being open to an area of the flash chamber that contains the biasing element.

2. The IV catheter device of claim 1, wherein the biasing element comprises a coil spring and the inner lumen of the actuatable obturator is axially aligned with a center of the coil spring.

3. The IV catheter device of claim 1, wherein the main housing has a distal end region that is received within the hollow needle hub and is adjacent a center region of the main housing that has increased width compared to the distal end region.

4. The IV catheter device of claim 1, wherein the actuatable obturator includes a hub that has a proximal end region, a center region and a distal end region, the center region having a greater width compared to both the distal end region and the proximal end region.

5. The IV catheter device of claim 4, wherein the proximal end region is received within one end of the biasing element and terminates at a location between opposite ends of the biasing element and the inner lumen extends through and is open at the proximal end of the obturator that comprises an end of the proximal end region that is at the location.

6. The IV catheter device of claim 1, further including an actuator for controllably operating the actuatable obturator, wherein the actuator moves between a raised position when the actuatable obturator is in the pre-deployed position and a lowered position when the actuatable obturator is in the deployed position.

7. The IV catheter device of claim 6, wherein the actuator is configured to permit the actuatable obturator to pass axially therethrough.

8. The IV catheter device of claim 6, wherein the actuator has a top portion accessible along an exterior of the main housing.

9. The IV catheter device of claim 1, wherein the obturator comprises an echogenic obturator that includes at least one echogenic element that provides localized increased echogenicity of the actuatable echogenic obturator and improved visualization of the actuatable echogenic obturator, wherein the actuatable echogenic obturator includes at least one region that is adjacent the at least one echogenic element and does not provide increased echogenicity.

10. The IV catheter device of claim 9, wherein the at least one echogenic element is located at the blunt distal end.

11. The IV catheter device of claim 9, wherein the at least one echogenic element comprises a first band and a second band spaced longitudinally from the first band.

12. The IV catheter device of claim 11, wherein each of the first band and the second band comprises one of an etched region of the actuatable echogenic obturator and a region that is formed of an echodense material.

13. The IV catheter device of claim 11, wherein in the fully deployed position, an entire width of at least a section of the at least one echogenic element is visible beyond the sharp distal tip of the elongated needle.

14. The IV catheter device of claim 1, further including a lock mechanism that is configured to maintain the actuatable obturator in the deployed position after the actuatable obturator has moved from the pre-deployed position to the deployed position.

15. The IV catheter device of claim 14, wherein the lock mechanism is automated and requires no user intervention to move from an initial state to an extended state in which the lock mechanism blocks the actuatable obturator and prevents movement of the actuatable obturator from the deployed position to the pre-deployed position.

16. The IV catheter device of claim 15, wherein the lock mechanism comprises a lock member that is biased by a spring, wherein in the initial state, the spring stores energy and the lock member seats against the actuatable obturator but does not prevent axial movement of the actuatable obturator within the main housing, while in the extended state, the lock member seats against a shoulder of the actuatable obturator to prevent the axial movement of the actuatable obturator in a proximal direction.

17. An IV catheter device comprising:
a needle component having an elongated needle with a sharp distal tip and a hollow needle housing from which the elongated needle extends in a distal direction, the hollow needle housing defining a flash chamber;
a catheter component having a hollow body through which the needle passes; and
an actuatable obturator that is disposed within the hollow needle housing and moves between a pre-deployment position and a deployed position, wherein in the pre-deployment position and the deployed position, the actuatable obturator is disposed within an inner lumen of the elongated needle and wherein in the deployed position, a blunt distal end of the obturator is located distal to the sharp distal tip so as to blunt the elongated needle;
wherein the actuatable obturator is biased by a biasing element that stores energy in the pre-deployment position and releases energy as the actuatable obturator moves from the pre-deployment position to the deployed position; the biasing element being disposed within the hollow needle housing within the flash chamber;
wherein the actuatable obturator is hollow and has an inner lumen defined therein that is open at proximal and distal ends of the obturator, the inner lumen at the proximal end of the obturator being open to an area of the flash chamber that contains the biasing element.

18. An IV catheter device comprising:
a needle component having an elongated needle with a sharp distal tip and a hollow needle hub from which the elongated needle extends;
a catheter component having a hollow body through which the needle passes;
a main housing that is coupled at one end to the hollow needle hub, the main housing defining a flash chamber; and
an actuatable obturator that is disposed within the main housing and moves between a pre-deployment position and a deployed position, wherein in the deployed position, the actuatable obturator is disposed within an inner lumen of the elongated needle and a blunt distal end of the obturator is located distal to the sharp distal tip so as to blunt the elongated needle;
wherein the actuatable obturator is biased by a biasing element that stores energy in the pre-deployment position and releases energy as the actuatable obturator moves from the pre-deployment position to the deployed position; the biasing element being disposed within the main housing within the flash chamber;
wherein the actuatable obturator is hollow and has an inner lumen formed therein that is open at proximal and distal ends of the obturator, the inner lumen at the proximal end of the obturator being open to an area of the flash chamber that contains the biasing element.

19. An IV catheter device comprising:
a needle component having an elongated needle with a sharp distal tip and a hollow needle housing from which the elongated needle extends in a distal direction, the hollow needle housing defining a flash chamber;
a catheter component having a hollow body through which the needle passes; and
an actuatable obturator that is disposed within the hollow needle housing and moves between a pre-deployment position and a deployed position, wherein in the pre-deployment position and the deployed position, the actuatable obturator is disposed within an inner lumen of the elongated needle and wherein in the deployed position, a blunt distal end of the obturator is located distal to the sharp distal tip so as to blunt the elongated needle;
wherein the actuatable obturator is biased by a biasing element that stores energy in the pre-deployment position and releases energy as the actuatable obturator moves from the pre-deployment position to the deployed position; the biasing element comprising a hollow coiled spring that is disposed within the hollow needle housing, the actuatable obturator having a hub portion that a proximal end region that is received internally within the biasing element;
an actuator coupled to the hollow needle housing and configured such that in a first position, the actuator blocks forward movement of the actuatable obturator and in a second position of the actuator, the actuatable obturator is free to move forward under force of the biasing element; and
a lock mechanism that is coupled to the hollow needle housing and moved between an unlocked position and a locked position, wherein in the pre-deployed position of the actuatable obturator, the lock mechanism is in the unlocked position and in the deployed position of the actuatable obturator, the lock mechanism is in the locked position.

20. The IV catheter device of claim 19, wherein the actuatable obturator is hollow and has an inner lumen defined therein that is open at proximal and distal ends of the obturator, the inner lumen at the proximal end of the obturator being open to an area of the flash chamber that contains the hollow coiled spring.

21. The IV catheter device of claim 19, wherein the actuator has a through hole through which the actuator obturator passes through, the actuator including a leg that is in contact with the actuatable obturator in the pre-deployed position, while the leg is displaced from the actuatable obturator in the deployed position, thereby permitting axial movement of the actuatable obturator through the through hole of the actuator.

22. The IV catheter device of claim 19, wherein the hub portion of the actuatable obturator includes a distal end region and a center region between the distal end region and the proximal end region, the center region having a diameter that is greater than a diameter of each of the proximal end region and the distal end region, wherein in the unlocked position, the lock mechanism is in contact with the center region of the hub portion.

23. The IV catheter device of claim 22, wherein in the locked position, the lock mechanism is disposed at a shoulder located between the center region and the proximal end region.

24. The IV catheter device of claim 4, wherein the biasing element comprises a coil spring and the proximal end region is received within one end of the coil spring such that the coil spring surrounds the proximal end region and the inner lumen is positioned inside the coil spring for allowing blood to flow directly into the flash chamber.
